(19) United States
(12) Patent Application Publication
(10) Pub. No.: US 2020/0131748 A1
(43) Pub. Date: Apr. 30, 2020

(54) Portable Toilet Odor Blocking Device and Toilet Odor Blocking Method

(71) Applicant: Lu-Ying Chiu, Kaohsiung City (TW)

(72) Inventor: Lu-Ying Chiu, Kaohsiung City (TW)

(21) Appl. No.: 16/668,591

(22) Filed: Oct. 30, 2019

(30) Foreign Application Priority Data
Oct. 31, 2018 (TW) 107138540

(51) Int. Cl. E03D 9/00; B05B 7/00; A61L 9/14

(52) U.S. Cl. CPC E03D 9/005; A61L 9/14; B05B 7/0018; E03D 9/007

(57) ABSTRACT

A portable toilet odor blocking device includes a container, a foaming liquid, a liquefied gas and a spraying module. The container is formed with an accommodation space. The foaming liquid is accommodated in the accommodation space. The liquefied gas is accommodated in the accommodation space. The spraying module is mounted to the container and configured to use air pressure of the liquefied gas to spray the foaming liquid to a water surface of a toilet, bowl and to selectively spray the foaming liquid to a wall surface of the toilet bowl, such that foam formed by the foaming liquid is configured to completely cover the water surface of the toilet bowl and selectively cover the wall surface of the toilet bowl. Wherein, the foam formed by the foaming liquid is configured to block an odor from excrement above or under the water surface of the toilet bowl.

### BACKGROUND OF THE INVENTION

### 1. Field of the Invention

The present invention relates to a portable toilet odor blocking device, and more particularly, to a portable toilet odor blocking device capable of reducing bad odor during using toilet.

### 2. Description of the Prior Art

Generally, excrement generates a bad odor during using toilet. Such odor not only makes people feel uncomfortable, but also makes people feel embarrassed after using the toilet. In order to solve the odor problem, a user usually uses an air freshener. However, a scent of the air freshener may be mixed with the odor of the excrement to generate weird smell. Therefore, the air freshener can not solve the odor problem during using the toilet.

### SUMMARY OF THE INVENTION

The present invention provides a portable toilet odor blocking device and a toilet odor blocking method.

The portable toilet odor blocking device comprises a container, a foaming liquid, a liquefied gas and a spraying module. The container is formed with an accommodation space. The foaming liquid is accommodated in the accommodation space. The liquefied gas is accommodated in the accommodation space. The spraying module is mounted to the container and configured to use air pressure of the liquefied gas to spray the foaming liquid to a water surface of a toilet bowl and to selectively spray the foaming liquid to a wall surface of the toilet bowl, such that foam formed by the foaming liquid is configured to completely cover the water surface of the toilet bowl and selectively cover the wall surface of the toilet bowl. The foam formed by the foaming liquid is configured to block an odor of excrement, above or under the water surface of the toilet bowl.

According to an embodiment of the portable toilet odor blocking device, the foaming liquid comprises a surfactant configured to generate foam.

According to an embodiment of the portable toilet odor blocking device, the foaming liquid further comprises a fragrant agent configured to be distributed in the air.

According to an embodiment of the portable toilet odor blocking device, the foaming liquid further comprises a deodorant configured to be distributed in the air for eliminating the odor of excrement.

According to an embodiment of the portable toilet odor blocking device, the foaming liquid is not corrosive.

The toilet odor blocking method comprises providing a portable container, wherein the portable container is filled with a foaming liquid and a liquefied gas; holding and moving the portable container to use air pressure of the liquefied gas to spray the foaming liquid to a water surface of a toilet bowl and to selectively spray the foaming liquid to a wall surface of the toilet bowl, such that foam formed by the foaming liquid is configured to completely cover the water surface of the toilet bowl and selectively cover the wall surface of the toilet bowl; and the foam formed by the foaming liquid blocking an odor from excrement above or under the water surface of the toilet bowl.

According to an embodiment of the toilet odor blocking method, the foaming liquid is sprayed to the toilet bowl before a user excreting to the toilet.

According to an embodiment of the toilet odor blocking method, the foaming liquid comprises a surfactant and a fragrant agent, and the portable toilet odor blocking method further comprises distributing the fragrant agent in the air during spraying the foaming liquid.

According to an embodiment of the toilet odor blocking method, the foaming liquid comprises a surfactant and a deodorant, and the portable toilet odor blocking method further comprises distributing the deodorant in the air to eliminate the odor of excrement in the air during spraying the foaming liquid.

According to an embodiment of the toilet odor blocking method, the foaming liquid is not corrosive.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram showing a portable toilet odor blocking device of the present invention;

FIG. 2 is a diagram showing the portable toilet odor blocking device of the present invention spraying a foaming liquid to a toilet bowl; and

FIG. 3 is a diagram showing foam formed by the foaming liquid being configured to block an odor of excrement.

### DETAILED DESCRIPTION

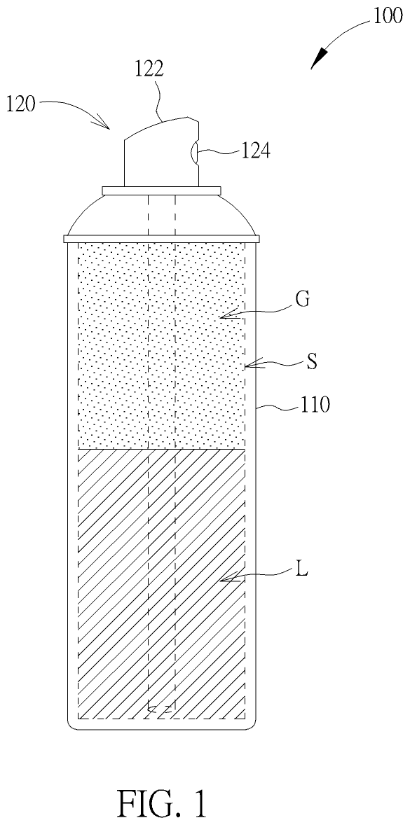

Please refer to FIG. 1. FIG. 1 is a diagram showing a portable toilet odor blocking device of the present invention. As shown in FIG. 1, the portable toilet odor blocking device 100 of the present invention comprises a container 110, a foaming liquid L, a liquefied gas G and a spraying module 120. The container 110 is formed with an accommodation space 5. The foaming liquid L and the liquefied gas G are accommodated in the accommodation space S. Some of the liquefied gas G is mixed with the foaming liquid L, and some of the liquefied gas G is vaporized within the accommodation space S. In the present invention, the liquefied gas G is butane, but the present invention is not limited thereto. The spraying module 120 is mounted to the container 110 and configured to use air pressure of the liquefied gas G to spray the foaming liquid L. For example, when a user presses a spraying head 122 of the spraying module 120, a nozzle 124 of the spraying module 120 is configured to communicate with the accommodation space S, so as to spray out the foaming liquid L by using the air pressure of the liquefied gas G. On the other hand, when the foaming liquid L is sprayed out by the spraying module 120, the liquefied gas G mixed in the foaming liquid L is also discharged with the foaming liquid L.

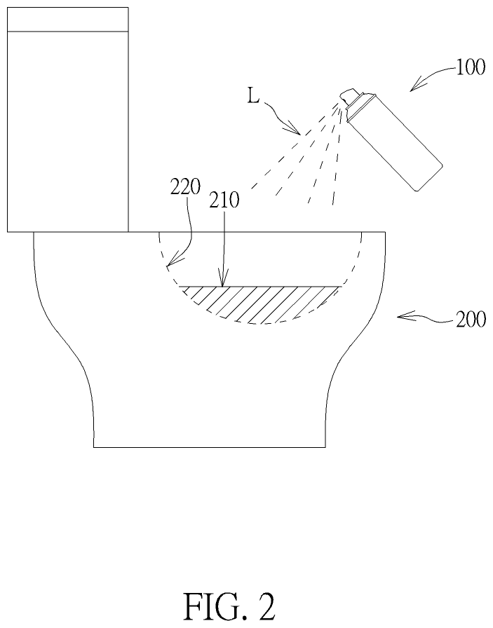

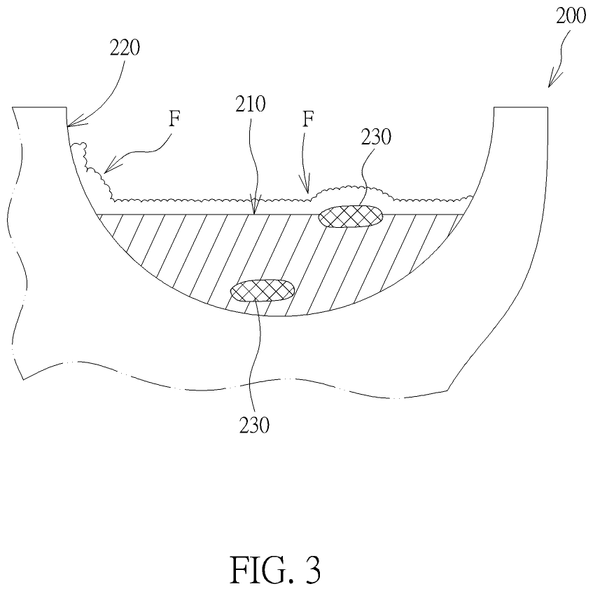

Please refer to FIG. 2 and FIG. 3, and refer to FIG. 1 as well. FIG. 2 is a diagram showing the portable toilet odor blocking device of the present invention spraying the foaming liquid to a toilet bowl. FIG. 3 is a diagram showing foam formed by the foaming liquid being configured to block an odor of excrement. As showing in figures, when using the portable toilet odor blocking device 100 of the present invention, the user can make the nozzle 124 of the spraying module 120 toward a water surface 210 of a toilet bowl 200. When the user holds the container 110 and presses the spraying head 122 of the spraying module 120, the spraying module 120 uses the air pressure of the liquefied gas G to spray the foaming liquid L to the water surface 210 of a toilet bowl 200, such that foam F formed by the foaming liquid L is configured to completely cover the water surface 210 of the toilet bowl 200. On the other hand, the user can also selectively spray the foaming liquid L to a wall surface 220 of the toilet bowl 200 by moving the container 110 such that foam F formed by the foaming liquid L is configured to selectively cover the wall surface 220 of the toilet bowl 200. Preferably the foaming liquid L is sprayed to the toilet bowl 200 before the user excreting to the toilet but the present invention is not limited thereto.

After the foam F formed by the foaming liquid L is sprayed to the toilet bowl 200 the foam F completely covers the water surface 210 of the toilet bowl 200. Therefore when the user excretes the foam F formed by the foaming liquid L is configured to block an odor from excrement 230 above or under the water surface 210 of the toilet bowl 200 in order to prevent the odor of the excrement 230 above or under the water surface 210 of the toilet bowl 200 from being distributed into the air. In other words the foam F formed by the foaming liquid L can isolate an odor source from the air. Therefore, the odor of the excrement 230 in the toilet bowl 200 cannot be distributed into the air.

In the present invention the foaming liquid h comprises a surfactant configured to generate the foam F. For example when the foaming liquid L is sprayed from the nozzle 124 of the spraying module 120 the foam F is generated by the surfactant due to agitation the gas. In addition, the foaming liquid L is not corrosive, so as to prevent the user from being harmed or feeling uncomfortable. Moreover the liquefied gas G mixed in the foaming liquid L is vaporized after being discharged such that air perturbation during vaporization of the liquefied gas G is increased, so as to improve generation of the foam F.

In another embodiment of the present invention, the foaming liquid L can further comprise a fragrant agent. When the foaming liquid L is sprayed from the nozzle 124 of the spraying module 120, the fragrant agent is configured to be distributed in the air in order to cover the odor in the air.

In another embodiment of the present invention, the foaming liquid L can further comprise a deodorant. When the foaming liquid L is sprayed from the nozzle 124 of the spraying module 120, the deodorant is configured to be distributed in the air, in order to eliminate the odor of the excrement in the air. For example, the deodorant can comprise ingredients, such as catechins, tannins, concentrated tannins, or chlorine dioxide, to neutralize or eliminate odor components of the excrement, (such as ammonia or hydrogen sulfide), but the present invention is not limited thereto, the foaming liquid L can comprise any kind of deodorant according to requirements.

According to the aforementioned arrangement, when the user excretes, the foam F formed by the foaming liquid L is configured to isolate the odor source from the air, and the fragrant agent in the foaming liquid L is configured to cover the odor in the air. In addition, the deodorant in the foaming liquid L is configured to eliminate the odor of the excrement in the air. Therefore, the portable toilet odor blocking device 100 of the present invention can effectively reduce the odor of the excrement when the user excretes, so as to solve the odor problem during using the toilet.

On the other hand, the foam F formed by the foaming liquid L is configured to selectively cover the wall surface 220 of the toilet bowl 200. Therefore, when the excrement falls onto the wall surface 220 of the toilet bowl 200, the foam F on the wall surface 220 is configured to allow the excrement to quickly slide into the water of the toilet bowl 200, such that the foam F on the water surface 210 can cover the excrement to prevent the odor from being distributed in the air. Moreover, the foam F on the wall surface 220 can also reduce possibility of the excrement being stuck onto the wall surface 220 of the toilet bowl 200.

The toilet, odor blocking method of the present invention comprises the following steps:
Step 10: Provide a portable container wherein the portable container is filled with a foaming liquid and a liquefied gas;
Step 20: Hold and move the portable container to use air pressure of the liquefied gas to spray the foaming liquid to a water surface of a toilet bowl and to selectively spray the foaming liquid to a wall surface of the toilet bowl, such that foam formed by the foaming liquid is configured to completely cover the water surface of the toilet bowl and selectively cover the wall surface of the toilet bowl; and
Step 30: The foam formed by the foaming liquid blocks an odor from excrement above or under the water surface of the toilet bowl by.

In contrast to the prior art, the portable toilet odor blocking device of the present invention can spray the foaming liquid to the toilet bowl, such that the foam formed by the foaming liquid can isolate the odor source from the air to solve the odor problem of during using the toilet. In addition, the foam formed by the foaming liquid can also reduce the possibility of the excrement being stuck onto the wall surface of the toilet bowl, in order to keep the toilet bowl clean.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A portable toilet odor blocking device, comprising:
a container formed with an accommodation space;
a foaming liquid accommodated in the accommodation space;
a liquefied gas accommodated in the accommodation space; and
a spraying module mounted to the container and configured to use air pressure of the liquefied gas to spray the foaming liquid to a water surface of a toilet bowl and to selectively spray the foaming liquid to a wall surface of the toilet bowl, such that foam formed by the foaming liquid is configured to completely cover the water surface of the toilet bowl and selectively cover the wall surface of the toilet bowl;
wherein the foam formed by the foaming liquid is configured to block an odor of excrement above or under the water surface of the toilet bowl.

2. The portable toilet odor blocking device of claim 1, wherein the foaming liquid comprises a surfactant configured to generate foam.

3. The portable toilet odor blocking device of claim 2, wherein the foaming liquid further comprises a fragrant agent configured to be distributed in the air.

4. The portable toilet odor blocking device of claim 2, wherein the foaming liquid further comprises a deodorant configured to be distributed in the air for eliminating the odor of excrement.

5. The portable toilet odor blocking device of claim 1, wherein the foaming liquid is not corrosive.

6. A toilet odor blocking method, comprising:
providing a portable container, wherein the portable container is filled with a foaming liquid and a liquefied gas;
holding and moving the portable container to use air pressure of the liquefied gas to spray the foaming liquid to a water surface of a toilet bowl and to selectively spray the foaming liquid to a wall surface of the toilet bowl, such that foam formed by the foaming liquid is configured to completely cover the water surface of the toilet bowl and selectively cover the wall surface of the toilet bowl; and
the foam formed by the foaming liquid blocking an odor from excrement above or under the water surface of the toilet bowl.

7. The toilet odor blocking method of claim 6, wherein the foaming liquid is sprayed to the toilet bowl before a user excreting to the toilet.

8. The toilet odor blocking method of claim 6, wherein the foaming liquid comprises a surfactant, and a fragrant agent, and the portable toilet odor blocking method further comprises distributing the fragrant agent in the air during spraying the foaming liquid.

9. The toilet odor blocking method of claim 6, wherein the foaming liquid comprises a surfactant and a deodorant, and the portable toilet odor blocking method further comprises distributing the deodorant in the air to eliminate the odor of excrement in the air during spraying the foaming liquid.

10. The toilet odor blocking method of claim 6, wherein the foaming liquid is not corrosive.
